(19) United States
(12) Patent Application Publication
(10) Pub. No.: US 2013/0317381 A1
(43) Pub. Date: Nov. 28, 2013

(54) WIRELESS DETECTION SYSTEM OF PHYSIOLOGICAL SIGNALS AND METHOD THEREOF

(71) Applicant: Taipei Medical University, Taipei City (TW)

(72) Inventors: Chung-che Wu, Taipei City (TW); Yung-hsiao Chiang, Taipei City (TW); Jung-tang Huang, Taipei City (TW)

(73) Assignee: Taipei Medical University, Taipei City (TW)

(21) Appl. No.: 13/901,101

(22) Filed: May 23, 2013

(30) Foreign Application Priority Data
May 25, 2012 (TW) 101118805

(51) Int. Cl. A61B 5/00

(52) U.S. Cl. CPC A61B 5/0031
USPC 600/544

(57) ABSTRACT

A wireless detection system of physiological signals is provided for wirelessly transmitting variation values of physiological signals, and includes a wireless detecting notification device and a wireless transceiver display device. The wireless detecting notification device comprises at least one transplantable sensor, a connecting line, a printed circuit board, a wireless communication micro-processor chip, an antenna and a power integration module. The power integration module includes a power management chip, a wireless charging coil and a chargeable battery, for integrating a power supply of the wireless detecting notification device. Further, a wireless detection method of physiological signals is further provided to detect variation values of physiological signals in organs of an organism. After calculating and analyzing the variation values of physiological signals, identifying an analyzed result for determining to store the variation values or to wirelessly transmit an alarm signal to the wireless transceiver display device.

### CROSS-REFERENCE

This application claims the priority of Taiwan Patent Application No. 101118805, filed on May 25, 2012.

### FIELD OF THE INVENTION

The present invention relates to a detection system and method of physiological signals and more particularly relates to a transplantable wireless detection system implanted into an organism to detect a variation value of physiological signals in an organ and a detection method thereof.

### BACKGROUND OF THE INVENTION

Traditionally, neurosurgical patients usually express signs of increased intracranial pressure (ICP), because their heads get serious impacts or have brain tumors. Even after operating craniotomy to patients, their intracranial pressure is persistently raised because hematoma or local tissue edema is performed to the region of the craniotomy, and then secondary brain injury (SBI) is accompanied to endanger their life. Therefore, the ICP detecting probe is used to be fixed into the skull of the critical patients who get serious impacts or are operated with craniotomy in neurosurgical intensive care unit (ICU), through drilling a burr hole, and that is to be an important index for caring and monitoring the living situation of those patients.

Referring to FIG. 1, the traditional ICP monitor for clinical usage is suitable for the critical patients in neurosurgical intensive care unit, because the probes 11, 12 of detecting the variation value of pressure signals must be implanted into the skull of these patients and a transmission line is used to connect to a pressure transducer (not shown). After the pressure transducer converts pressure signals detected by the probes 11, 12 into electrical signals, a transmission line is used to transmit the electrical signals to a display of a physiological monitor (not shown) for showing pressure waves. In other words, a whole set of the pressure detecting system, from the probes 11, 12 implanted into the brain of the patient to the shown pressure waves, not only has several of large medical devices connected to each other in series, but also connecting lines and transmission lines must be used to be the mediators of signal transmission. As a result, the body movement of the patient is limited, and the pressure detecting system is also not suitably applied to long-term monitor the home care of patients.

Recently, with the development of wireless transmission bio-chips and the maturation of Micro-Electro Mechanical Systems (MEMS) technologies, the miniaturization of medical devices and the wireless signal transmission become the major direction of research and development in present related industries. For example, the University of Washington in America proposes a passive ICP monitor, wherein the characteristic thereof is to mount a signal receiver adjacent to a pressure monitor for detecting the values of intracranial pressure through skins. Although the design of the ICP monitor is conformed with the intention of wireless transmission, its signals are easily interfered by others electro-magnetic waves in the environments. Therefore, it is difficult to apply the ICP monitor to the remote monitoring.

As a result, the traditional transplantable sensor for wireless transmission is still not suitable for being applied to the clinic, therefore, it is necessary to provide a wireless detection system of physiological signals and a method thereof to solve the problems existing in the conventional technologies.

### SUMMARY OF THE INVENTION

A primary object of the present invention is to provide a wireless detection system of physiological signals and a method thereof, which uses a wireless detecting notification device to detect and analyze a variation value of physiological signals in an organ of an organism, uses an antenna to transmit an alarm, or uses a wireless transceiver display device to receive a download instruction transmitted from a remote end, for ensuring to decrease the influence of health of the organism after the device is implanted to the organism (such as a clinical patient) and persistently transmit wireless signals. Simultaneously, the wireless detection system can be used for long-term monitoring physiological situations to the patient dependent on the needs of pathological status, and also can efficiently solve the conventional clinic problem that the transplantable probe implanted into the brain of neurosurgical patient causes an open wound on the localized part of the head to increase the infection rate to the brain of the patient and to induce second brain injury. Therefore, it is advantageous to increase the safety of the wireless transmission device applied for monitoring pathological brain injury.

A secondary object of the present invention is to provide a wireless detection system of physiological signals and a method thereof, which uses a micro-processor, which is a low-power Bluetooth chip of system-on-a-chip (SOC), to calculate, analyze and store a variation value of physiological signals in an organism, so as to simplify the large medical equipment or display, connected for monitoring the variation value of physiological signals in the conventional clinic for the purpose of achieving the miniaturization design in the medical equipment, so that it is more convenient to monitor and record the variation values of physiological signals in various organs. Therefore, it is actually suitable for the patient who needs to be monitored the living situation of physiological signals in the organ for a long time.

A further object of the present invention is to provide a wireless detection system of physiological signals and a method thereof, wherein the wireless transceiver display device can be a transceiver device for receiving an alarm signal transmitted from the wireless detecting notification device and transmitting the alarm signal to a remote medical display; or it also can be a multi-functional transceiver device for receiving the alarm signal, transmitting the remote instruction, and analyzing, displaying or storing transmitted data, so that it is convenient to connect to various intelligent electrical devices, which can support cloud transmission. Therefore, it is advantageous to increase the convenience for remote monitoring and health care, and extent the wireless transmission range of the system.

To achieve the above object, the present invention provides a wireless detection system of physiological signals, which comprises:

a wireless detecting notification device implanted onto an organ of an organism to detect a variation value of physiological signals of the organ, calculate and analyze the variation value of physiological signals, and then select to store a normal physiological value or to wirelessly transmit an alarm signal; and a wireless transceiver display device used to wirelessly receive the normal physiological value or the alarm signal from the wireless detecting notification device, or to wireless transmit a remote instruction to the wireless detecting notification device, so that the wireless detecting notification device wirelessly transmits the alarm signal or the normal physiological value to the wireless transceiver display device for analyzing and storing.

In one embodiment of the present invention, the wireless detecting notification device comprises:

at least one transplantable sensor implanted onto the organ for detecting the variation value of physiological signals, which is generated from the organ;

a connecting line having one end electrically connected to the transplantable sensor for transmitting the detected variation value of physiological signals;

a printed circuit board electrically connected to the other end of the connecting line for being a mediator to transmit the variation value of physiological signals;

a wireless communication micro-processor chip mounted on the printed circuit board for calculating and analyzing the variation value of physiological signals, which is transmitted from the printed circuit board, so as to obtain an analyzed result and then determine to actively transmit the alarm signal or only store as the normal physiological value according the analyzed result;

an antenna mounted on the printed circuit board for wirelessly transmitting the alarm signal or the normal physiological value, or receiving the remote instruction from the wireless transceiver device; and

a power integration module comprising a power management chip, a wireless charging coil and a chargeable battery, and used to integrate electric power supplement of the wireless detecting notification device.

In one embodiment of the present invention, the wireless transceiver display device is a cloud system, a personal computer, a laptop computer, a tablet computer, a palmtop computer, a digital watch or a smartphone.

In one embodiment of the present invention, the connecting line is a bio-compatible connecting line.

In one embodiment of the present invention, the bio-compatible connecting line has an outer coating made of silicone material.

In one embodiment of the present invention, the wireless communication micro-processor chip is a low-power Bluetooth chip of system-on-a-chip (SOC).

In one embodiment of the present invention, the variation value of physiological signals is a variation value of organic pressure, blood flow, heart rate, body temperature, respiration rate or electrical current.

Furthermore, to achieve the above object, the present invention further provides a wireless detection method of physiological signals, which comprises steps of:

implanting a wireless detecting notification device onto an organ of an organism;

using at least one transplantable sensor of the wireless detecting notification device to detect a variation value of physiological signals in the organ;

using a wireless communication micro-processor chip of the wireless detecting notification device to calculate and analyze the variation value of physiological signals for obtaining an analyzed result; and

using the wireless communication micro-processor chip to identify the analyzed result for selecting to store a normal physiological value or wirelessly transmit an alarm signal.

In one embodiment of the present invention, the step of identifying the analyzed result by the wireless communication micro-processor chip comprises steps of:

using the wireless communication micro-processor chip to identify if the analyzed result is the alarm signal;

if yes, immediately transmitting the alarm signal to an antenna, and actively wirelessly transmitting the alarm signal to the transceiver display device; and

if not, identifying the analyzed result to be a normal physiological signal, and storing the normal physiological signal to the wireless communication micro-processor chip.

In one embodiment of the present invention, after the step of storing the normal physiological signal, further comprising a step of:

using the antenna to transmit the normal physiological signal to the wireless transceiver display device when receiving a remote instruction from the wireless transceiver display device.

In one embodiment of the present invention, after the step of identifying the analyzed result by the wireless communication micro-processor chip, further comprising a step of:

using the wireless transceiver display device to transmit a remote instruction to the wireless detecting notification device, so that the wireless detecting notification device wirelessly transmits the alarm signal or the normal physiological signals to the wireless transceiver display device for analyzing and storing.

### DESCRIPTION OF THE DRAWINGS

FIG. 1 is a schematic view of a conventional detection method of intracranial pressure (ICP);

FIG. 2 is a schematic view of a wireless detection system of physiological signals according to a preferred embodiment of the present invention;

FIG. 3A is a top view of a wireless detecting notification device according to the preferred embodiment of the present invention;

FIG. 3B is a bottom view of the wireless detecting notification device according to the preferred embodiment of the present invention;

FIG. 3C is a side view of the wireless detecting notification device according to the preferred embodiment of the present invention;

FIG. 4 is an operational view of a wireless transceiver display device according to the preferred embodiment of the present invention; and

FIGS. 5A, 5B are block diagrams of the wireless detection method of physiological signals according to the preferred embodiment of the present invention.

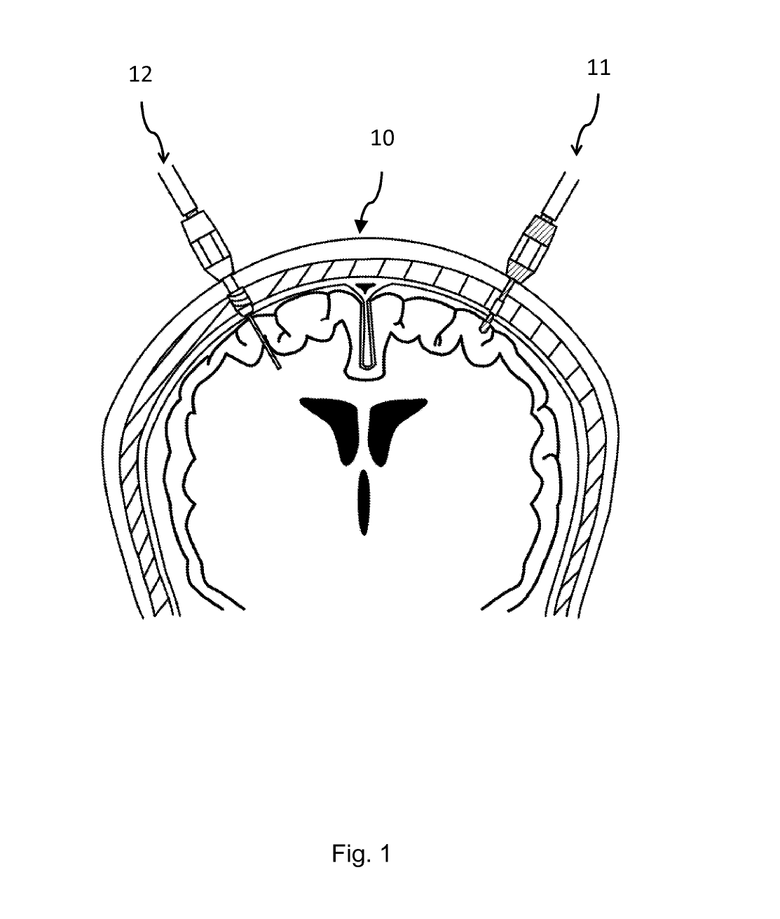

### DETAILED DESCRIPTION OF THE PREFERRED EMBODIMENTS

The structure and the technical means adopted by the present invention to achieve the above and other objects can be best understood by referring to the following detailed description of the preferred embodiments and the accompanying drawings. Furthermore, directional terms described by the present invention, such as upper, lower, front, back, left, right, inner, outer, side, longitudinal/vertical, transverse/horizontal, and etc., are only directions by referring to the accompanying drawings, and thus the used directional terms are used to describe and understand the present invention, but the present invention is not limited thereto.

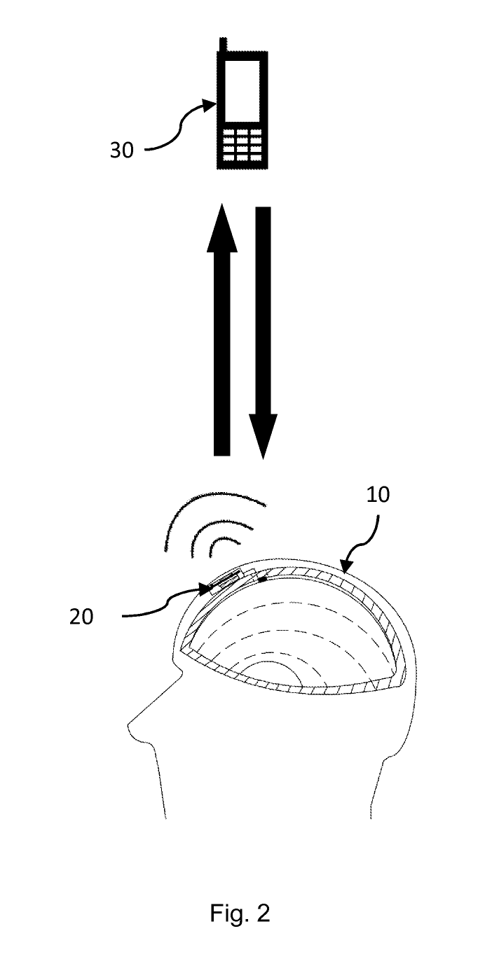

Referring to FIG. 2, a wireless detection system of physiological signals according to a preferred embodiment of the present invention is illustrated. As shown, the wireless detection system comprises a wireless detecting notification device 20 and a wireless transceiver display device 30. The wireless detecting notification device 20 is implanted onto an organ of an organism to detect a variation value of physiological signals of the organ, wherein the variation value of physiological signals is a variation value of organic pressure, blood flow, heart rate, body temperature, respiration rate or electrical current. For example, in the present embodiment, the wireless detecting notification device 20 is implanted into a skull 10 for detecting an intracranial pressure (ICP) and a variation value of physiological signals, wherein after calculating and analyzing the variation value of physiological signals, the wireless detecting notification device 20 can select to store a normal physiological value or to wirelessly transmit an alarm signal. The wireless transceiver display device 30 is used to wirelessly receive the normal physiological value or the alarm signal from the wireless detecting notification device 20, or to wirelessly transmit a remote instruction to the wireless detecting notification device 20, so that the wireless detecting notification device 20 wirelessly transmits the alarm signal or the normal physiological value to the wireless transceiver display device 30 for analyzing and storing.

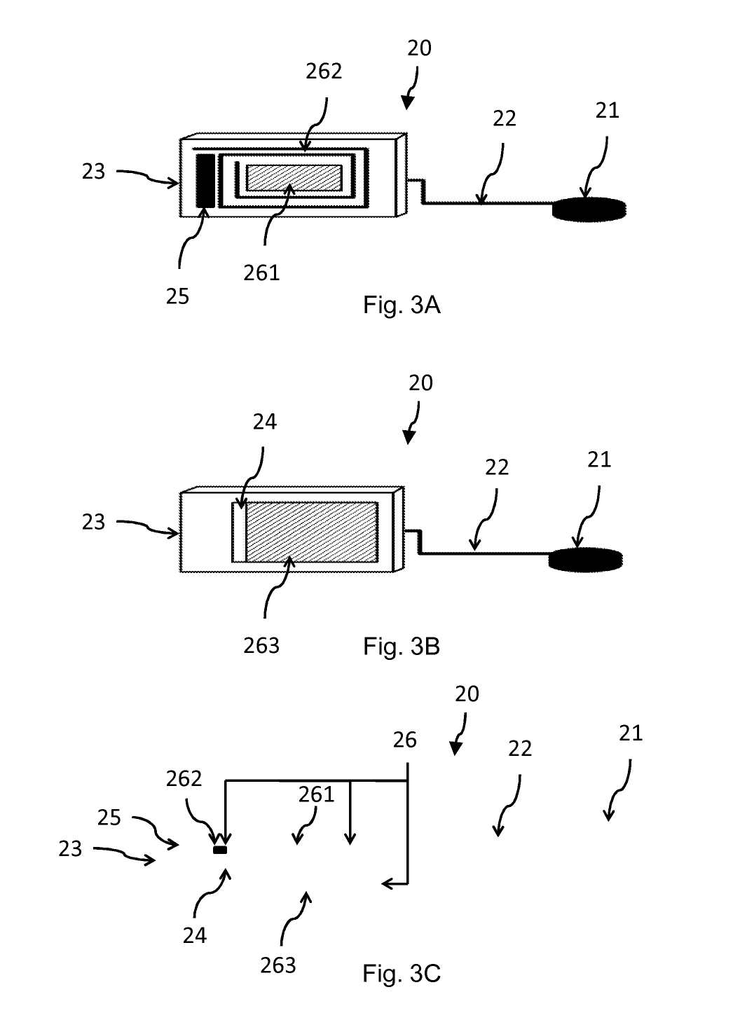

Referring to FIGS. 3A, 3B and 3C, the wireless detection system of physiological signals according to the preferred embodiment of the present invention is illustrated, wherein the wireless detecting notification device 20 comprises at least one transplantable sensor 21, a connecting line 22, a printed circuit board 23, a wireless communication micro-processor chip 24, an antenna 25 and a power integration module 26.

Referring to FIG. 2 and FIGS. 3A to 3C again, the transplantable sensor 21 is electrically connected to one end of the connecting line 22, the other end of the connecting line 22 is electrically connected to the printed circuit board 23. In the present embodiment, the transplantable sensor 21 is exemplified by an intracranial pressure sensor implanted into a skull of a patient is for neurosurgery operating. Firstly, it has to operate craniotomy on the patient skull for placing the transplantable sensor 21 and a partial of the connecting line 22 into the skull within the dura mater. Further, the residual part of the connecting line 22 and the other components of the wireless detecting notification device 20 are placed between the skull and the subcutaneous tissues of the head, and then the subcutaneous tissues of the head are completely sutured. Therefore, the wireless detecting notification device 20 is can be stably placed into the skull 10 of the patient for detecting the variation value of physiological signals under the skull. Because the wireless detecting notification device 20 is placed into the subcutaneous tissue of the head through a sterilized suture surgery without contacting to outside environments through other wires, it thus decreases the infection problem while the patient uses the wireless detection system for long-term monitoring.

Furthermore, the connecting line 22 is preferably the bio-compatible connecting line which has an outer coating made of silicone material, but not limited thereto. One end of the connecting line 22 is electrically connected to the transplantable sensor 21 for transmitting detected the variation signals of intracranial pressure. Except for the transplantable sensor 21 and the connecting line 22, the other components of the wireless detecting notification device 20 are packaged within a bio-compatible material (not shown), wherein the package material is preferably silicon material, but not limited thereto.

Referring to FIGS. 3A to 3C again, the printed circuit board 23 according to the preferred embodiment of the present invention is electrically connected to the other end of the connecting line 22 and is used to be a mediator for transmitting the variation value of physiological signals. The printed circuit board 23 has a first surface and a second surface. The first surface carries the wireless communication micro-processor chip 24 and a portion of the power integration module 26 in turn. Simultaneously, the second surface carries the antenna 25 and the other portion of the power integration module 26. Moreover, the wireless communication micro-processor chip 24 is preferably a low-power Bluetooth chip of system-on-a-chip (SOC), such as TI Bluetooth 4.0 chip of CC2540, but not limited thereto. In the present embodiment, the wireless detecting notification device 20 mainly uses the wireless communication micro-processor chip 24 of low-power Bluetooth to be a component for analyzing and storing signals, so that the wireless detecting notification device 20 can persist for long-term operating in the relatively low electric power consumption.

Additionally, the antenna 25 is mounted on the printed circuit board 23, for wirelessly transmitting an alarm signal through Bluetooth, transmitting a normal physiological value stored at the wireless communication micro-processor chip 24, or receiving a remote instruction transmitted from the wireless transceiver display device 30. Therefore, the distance, in which the wireless detecting notification device 20 can receive or transmit the signals, is not limited to a general transmission line, wherein the distance can achieve 10 to 50 meters (m) for transmitting the signals. In the present embodiment, the wireless communication micro-processor chip 24 can analyze the detected intracranial pressure signals. While the wireless communication micro-processor chip 24 identifies that the detected intracranial pressure achieves to a dangerous value, it then transmits a signal to the antenna 25, the antenna 25 further wirelessly transmits an alarm signal to the wireless transceiver display device 30; if the wireless communication micro-processor chip 24 identifies that the detected intracranial pressure is a normal physiological value, it then stores the signal value to the communication micro-processor chip 24 directly. Only if the antenna 25 receives a remote instruction, the stored data is wirelessly transmitted to the wireless transceiver display device 30 for analyzing and storing the data.

Furthermore, the power integration module 26 comprises a power management chip 261, a wireless charging coil 262 and a chargeable battery 263, and it uses to integrate the electric power supplement of the wireless detecting notification device 20. The power management chip 261 is mounted on the second surface of the printed circuit board 23, and surrounded by the wireless charging coil 262, wherein the wireless charging coil 262 can transmit electrical power to the power management chip 261, and also can be used for wirelessly charging. The chargeable battery 263 is mounted on the wireless communication micro-processor device 24 on the first surface of the printed circuit board 23 (or other position of the first surface). The power integration module 26 is mainly used to serially integrate the electric power supplement of the wireless detecting notification device 20, for example, the necessary electrical power for the transplantable sensor 21 detecting the physiological value, the wireless communication micro-processor chip 24 used to analyze and store, and the antenna 26 operating to transmit is all supplied by the power integration module 26.

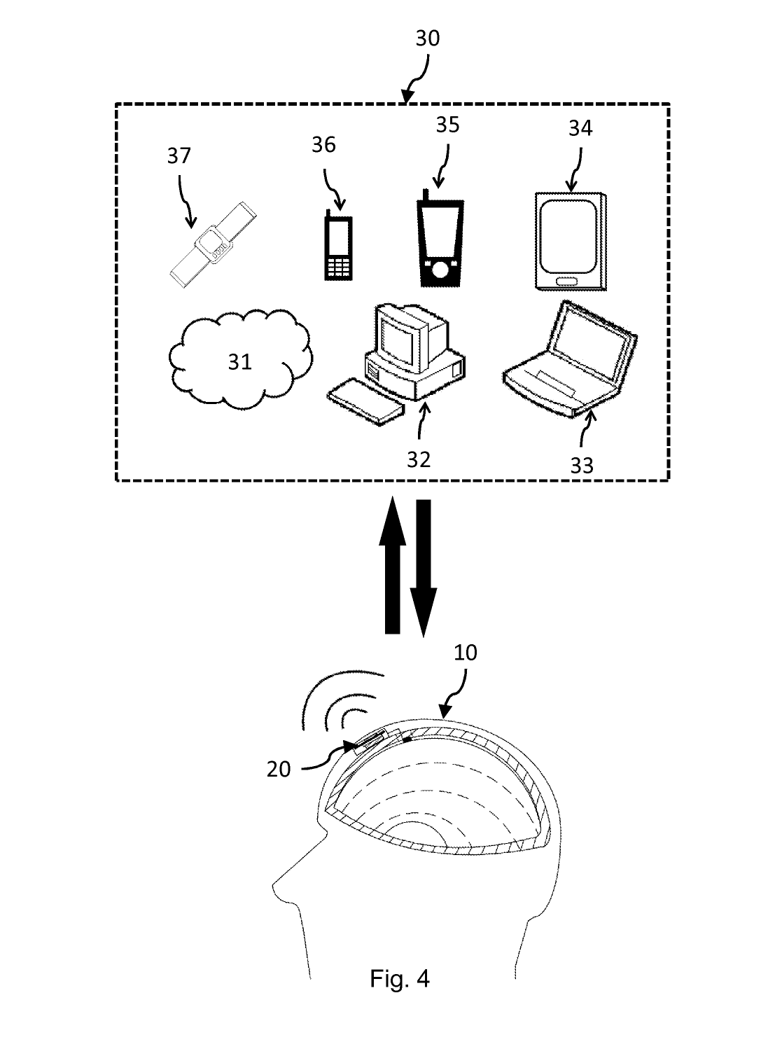

Referring to FIG. 4, the wireless transceiver display device 30 can select from a cloud system 31, a personal computer 32, a laptop computer 33, a tablet computer 34, a palmtop computer 35, a digital watch 36 or a smartphone 37, but not limits thereto. Further, the wireless transceiver display device 30 is used for receiving the alarm signal transmitted from the wireless detecting notification device 20, storing and analyzing the data transmitted from the device, or wirelessly transmitting a remote instruction to the wireless detecting notification device 20.

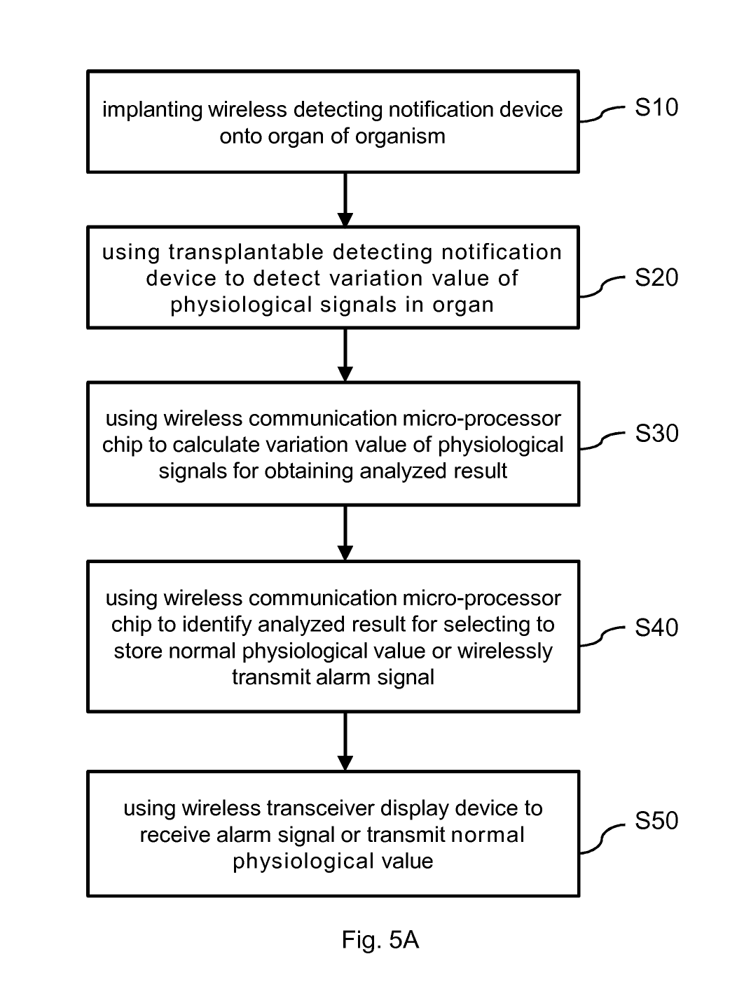

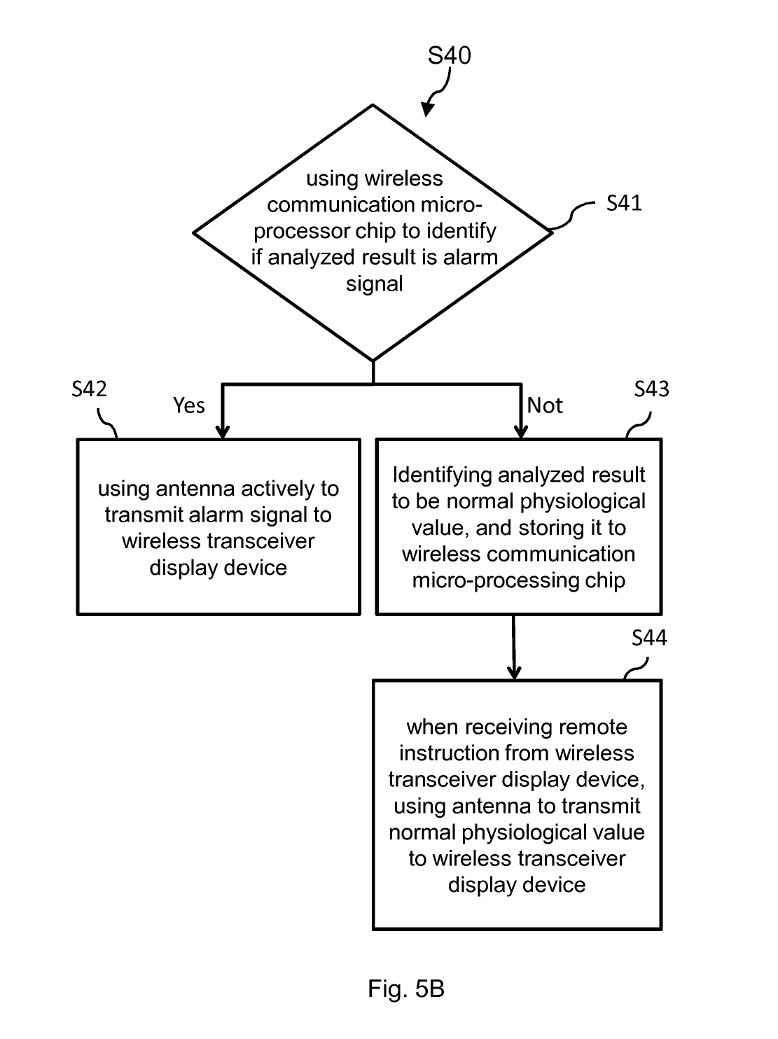

Referring to FIG. 5, in the preferred embodiment of the present invention, according to the wireless detection system of physiological signals, as described above, the present invention provides a wireless detection method of physiological signals, which comprises steps of: (S10) implanting a wireless detecting notification device 20 onto an organ of an organism (such as a skull of human); (S20) using at least one transplantable sensor 21 of the wireless detecting notification device 20 to detect a variation value of physiological signals of the organ; (S30) using a wireless communication micro-processor chip 24 of the wireless detecting notification device 20 to calculate and analyze the variation value of physiological signals for obtaining an analyzed result; and (S40) using the wireless communication micro-processor chip 24 to identify the analyzed result for selecting to store a normal physiological value or wirelessly transmit an alarm signal.

Moreover, referring to FIG. 5A, the step (S40) of identifying the analyzed result by the wireless communication micro-processor chip 24 comprises steps of: (S41) using the wireless communication micro-processor chip 24 to identify if the analyzed result is the alarm signal; (S42) if yes, immediately transmitting the alarm signal to an antenna 25, and actively wirelessly transmitting the alarm signal to the wireless transceiver display device 30; and (S43) if not, identifying the analyzed result to be a normal physiological signal, and storing the normal physiological signal to the wireless communication micro-processor chip 24. And, after the step (S43) of storing the normal physiological signal, it further comprises a step of: (S44) using the antenna 25 to wirelessly transmit the normal physiological signals to the wireless transceiver display device 30 when receiving a remote instruction from the wireless transceiver display device 30. After the normal physiological value is stored, it is not actively transmitted to the wireless transceiver display device 30, so that the operation of the wireless detecting notification device 20 for detecting, transmitting and storing the physiological signals only needs to consume extra-low electrical power. Thus, the electric capability of the chargeable battery 263 is enough for the wireless detecting notification device 20 to maintain the operation for a long time.

Additionally, referring to FIG. 5B, after the step (S40) of identifying the analyzed result by the wireless communication micro-processor chip 24, it further comprises a step of: (S50) using the wireless transceiver display device 30 to transmit a remote instruction to the wireless detecting notification device 20, so that the wireless detecting notification device 20 wirelessly transmits the alarm signal or stores the normal physiological signals to the wireless transceiver display device 30 for analyzing and storing. In the embodiment, the remote instruction is from a medical organization or a medical staff, which transmits the remote instruction to download the data stored by the wireless communication micro-processor chip 24, and then the stored data is copied for backup storage or medical analysis.

As described above, in comparison with the conventional transplantable probe of the ICP monitor for the brain of a neurosurgical patient, which easily generates open wound on the skull of the patient to increase the infection rate of the patient, the wireless detecting notification device 20 according to the present invention is implanted into the subcutaneous tissues of the head or other organs for the patient and the wound can be sutured completely, so that the wireless detecting notification device 20 can be used to detect and analyze the variation value of physiological signals those organs. Further, after receiving a remote instruction for downloading the data, the antenna 25 then transmits the data to the wireless transceiver display device 30 for analyzing or storing. Therefore, the invention can decrease the infection rate when the patient is monitored the situation of physiological signals in a long time period, and also decrease the harmful influence of electromagnetic waves persistently transmitted for a long time to the injury organ of the patient. For this reason, it can enhance the safety of long-term wirelessly transmission for the patient with pathological injury of the organ.

Furthermore, the present invention uses the wireless communication micro-processor device 24, which is a low-power Bluetooth chip of system-on-a-chip (SOC), to calculate, analyze and store the variation value of physiological signals to the organ of the organism. That is to apply Micro-Electro Mechanical Systems (MEMS) technologies to improve the conventional large medical devices or display used to monitor various variation values of physiological signals, so as to achieve the miniaturized design on the medical devices. As a result, the wireless detecting system of physiological signals according to the present invention provides more convenience in clinical application to human, and the system is suitable for the patient that needs to be monitored various variation values of physiological signals in the body.

Additionally, the wireless transceiver display device 30 can be a transceiver device to receive the alarm signal transmitted from the wireless detecting notification device 20, transmit the alarm signal to a display in remote end; or be a multi-functional transceiver device to receive the alarm signal, transmit the remote instruction and display the transmitted data, so that it is convenient to connect various intelligent electrical devices supporting cloud transmission. Therefore, the wireless detecting notification device 20 cooperated with the wireless transceiver display device 30 has advantages to increase the convenience of the wireless detecting system of physiological signals for the remote monitoring and to extend the wireless transmission range thereof.

The present invention has been described with a preferred embodiment thereof and it is understood that many changes and modifications to the described embodiment can be carried out without departing from the scope and the spirit of the invention that is intended to be limited only by the appended claims.

1. A wireless detection system of physiological signals, comprising:
a wireless detecting notification device implanted onto an organ of an organism to detect a variation value of physiological signals of the organ, calculate and analyze the variation value of physiological signals, and then select to store an normal physiological value or to wirelessly transmit an alarm signal; and
a wireless transceiver display device used to wirelessly receive the normal physiological value or the alarm signal from the wireless detecting notification device, or to wirelessly transmit a remote instruction to the wireless detecting notification device, so that the wireless detecting notification device wirelessly transmits the alarm signal or the normal physiological value to the wireless transceiver display device for analyzing and storing.

2. The wireless detection system of physiological signals and method according to claim 1, wherein the wireless detecting notification device comprises:
at least one transplantable sensor implanted onto the organ for detecting the variation value of physiological signals, which is generated from the organ;
a connecting line having one end electrically connected to the transplantable sensor for transmitting the detected variation value of physiological signals;
a printed circuit board electrically connected to the other end of the connecting line for being a mediator to transmit the variation value of physiological signals;
a wireless communication micro-processor chip mounted on the printed circuit board for calculating and analyzing the variation value of physiological signals, which is transmitted from the printed circuit board, so as to obtain an analyzed result and then determine to actively transmit the alarm signal or only store as the normal physiological value according the analyzed result;
an antenna mounted on the printed circuit board for wirelessly transmitting the alarm signal or the normal physiological value, or receiving the remote instruction from the wireless transceiver device; and
a power integration module comprising a power management chip, a wireless charging coil and a chargeable battery, and used to integrate electric power supplement of the wireless detecting notification device.

3. The wireless detection system of physiological signals and method according to claim 1, wherein the wireless transceiver display device is a cloud system, a personal computer, a laptop computer, a tablet computer, a palmtop computer, a digital watch or a smartphone.

4. The wireless detection system of physiological signals and method according to claim 2, wherein the connecting line is a bio-compatible connecting line.

5. The wireless detection system of physiological signals and method according to claim 4, wherein the bio-compatible connecting line has an outer coating made of silicone material.

6. The wireless detection system of physiological signals and method according to claim 2, wherein the wireless communication micro-processor chip is a low-power Bluetooth chip of system-on-a-chip (SOC).

7. The wireless detection system of physiological signals and method according to claim 1, wherein the variation value of physiological signals is a variation value of organic pressure, blood flow, heart rate, body temperature, respiration rate or electrical current.

8. A wireless detection method of physiological signals, comprising steps of:
implanting a wireless detecting notification device onto an organ of an organism;
using at least one transplantable sensor of the wireless detecting notification device to detect a variation value of physiological signals in the organ;
using a wireless communication micro-processor chip of the wireless detecting notification to calculate and analyze the variation value of physiological signals for obtaining an analyzed result; and
using the wireless communication micro-processor chip to identify the analyzed result for selecting to store a normal physiological value or wirelessly transmit an alarm signal.

9. The wireless detection method of physiological signals according to claim 8, wherein the step of identifying the analyzed result by the wireless communication micro-processor chip comprises steps of:
using the wireless communication micro-processor chip to identify if the analyzed result is the alarm signal;
if yes, immediately transmitting the alarm signal to an antenna, and actively wirelessly transmitting the alarm signal to the transceiver display device; and
if not, identifying the analyzed result to be a normal physiological signal, and storing the normal physiological signal to the wireless communication micro-processor chip, wherein when receiving a remote instruction from the wireless transceiver display device, using the antenna to transmit the normal physiological signal to the wireless transceiver display device.

10. The wireless detection method of physiological signals according to claim 8, wherein after the step of identifying the analyzed result by the wireless communication micro-processor chip, further comprising a step of:
using the wireless transceiver display device to transmit a remote instruction to the wireless detecting notification device, so that the wireless detecting notification device wirelessly transmits the alarm signal or the normal physiological signals to the wireless transceiver display device for analyzing and storing.
